(19) United States
(12) Patent Application Publication
(10) Pub. No.: US 2025/0057467 A1
(43) Pub. Date: Feb. 20, 2025

(54) THREE-DIMENSIONAL GAIT SIGNAL-BASED GERIATRIC COGNITIVE IMPAIRMENT DIAGNOSIS DEVICE AND METHOD

(71) Applicant: SEOUL NATIONAL UNIVERSITY R&DB FOUNDATION, Seoul (KR)

(72) Inventor: Kyoung-Bok MIN, Seoul (KR)

(73) Assignee: SEOUL NATIONAL UNIVERSITY R&DB FOUNDATION, Seoul (KR)

(21) Appl. No.: 18/937,164

(22) Filed: Nov. 5, 2024

Related U.S. Application Data
(63) Continuation of application No. PCT/KR2023/007520, filed on Jun. 1, 2023.

(30) Foreign Application Priority Data
Sep. 29, 2022 (KR) 10-2022-0124311

(51) Int. Cl. A61B 5/00; A61B 5/11; G16H 50/20

(52) U.S. Cl. CPC A61B 5/4088; A61B 5/112; A61B 5/1121; A61B 5/7271; G16H 50/20

(57) ABSTRACT

A three-dimensional gait signal-based geriatric cognitive impairment diagnosis device includes a data transmission/reception module; a memory storing a geriatric cognitive impairment diagnosis program; and a processor configured to execute the geriatric cognitive impairment diagnosis program stored in the memory, herein the geriatric cognitive impairment diagnosis program collects a three-dimensional gait signal including a spatiotemporal gait signal and a kinematic gait signal of an examinee, calculates a cognitive disorder disease probability value by using a cognitive disorder diagnosis model based on the three-dimensional gait signal, and determines whether the examinee has a cognitive disorder disease based on the calculated cognitive disorder disease probability value, and the cognitive disorder diagnosis model is constructed by applying the three-dimensional gait signal of a patient with a geriatric cognitive disorder disease to a logistic function.

### CROSS-REFERENCE TO RELATED APPLICATION

Pursuant to 35 USC 120 and 365(c), this application is a continuation of International Application No. PCT/KR2023/007520 filed on Jun. 1, 2023, and claims the benefit under 35 USC 119(a) of Korean Patent Application No. 10-2022-0124311 filed in the Korean Intellectual Property Office on Sep. 29, 2022, the entire disclosures of which are incorporated herein by reference for all purposes.

### BACKGROUND

The present disclosure relates to a three-dimensional gait signal-based geriatric cognitive impairment diagnosis device and method.

Dementia is a representative neurological disease caused by aging. The proportion of the elderly population in Korea is currently much higher than the proportion of the elderly population worldwide, and the incidence of dementia and mild cognitive impairment, which are degenerative geriatric neurological diseases, is expected to increase rapidly in the future. Conventional geriatric cognitive impairment (dementia and mild cognitive impairment) diagnosis technologies have limitations in that the technologies are excessively expensive or invasive due to the use of expensive imaging diagnostic equipment. In addition, questionnaire-based psychological tests have limitations in that the psychological tests are limited to measuring psychological phenomena rather than medical aspects.

Recently, research results showing a high relationship between geriatric cognitive impairment and gait have been presented, and a geriatric cognitive impairment prediction method using this is being studied.

In this regard, Korean Patent No. 10-2102931 (Title of the Invention: DEMENTIA PREDICTION SYSTEM) discloses a dementia prediction system that generates dementia prediction information based on gait speed calculated from the data received from a sensor device.

### SUMMARY

The present disclosure diagnoses geriatric cognitive impairment early based on measurement and evaluation of three-dimensional gait signals and indices including spatiotemporal gait signals and kinematic gait signals.

However, technical tasks to be achieved by the present embodiment are not limited to the technical task described above, and there may be other technical tasks.

According to an aspect of the present disclosure, a three-dimensional gait signal-based geriatric cognitive impairment diagnosis device includes a data transmission/reception module; a memory storing a geriatric cognitive impairment diagnosis program; and a processor configured to execute the geriatric cognitive impairment diagnosis program stored in the memory, herein the geriatric cognitive impairment diagnosis program collects a three-dimensional gait signal including a spatiotemporal gait signal and a kinematic gait signal of an examinee, calculates a cognitive disorder disease probability value by using a cognitive disorder diagnosis model based on the three-dimensional gait signal, and determines whether the examinee has a cognitive disorder disease based on the calculated cognitive disorder disease probability value, and the cognitive disorder diagnosis model is constructed by applying the three-dimensional gait signal of a patient with a geriatric cognitive disorder disease to a logistic function.

According to another aspect of the present disclosure, a three-dimensional gait signal-based geriatric cognitive impairment diagnosis method includes collecting a three-dimensional gait signal including a spatiotemporal gait signal and a kinematic gait signal of an examinee; calculating a cognitive impairment disease probability value by using a cognitive impairment diagnosis model based on the three-dimensional gait signal; and determining whether the examinee has a cognitive impairment disease based on the calculated cognitive impairment disease probability value, wherein the cognitive impairment diagnosis model is constructed by applying a three-dimensional gait signal of a patient with a geriatric cognitive impairment disease to a logistic function.

According to the present disclosure, by quantifying and synthesizing the measurement of kinematic analysis and spatiotemporal gait analysis in response to a three-dimensional gait signal, a device and system may diagnose a cognitive impairment risk group at an early stage and diagnose more accurately than the known gait analysis dementia diagnosis devices.

### BRIEF DESCRIPTION OF THE DRAWINGS

Embodiments of the inventive concept will be more clearly understood from the following detailed description taken in conjunction with the accompanying drawings in which:

FIG. 1 is a configuration diagram of a three-dimensional gait signal-based geriatric cognitive impairment diagnosis device according to an embodiment of the present disclosure;

FIG. 2 is a structural diagram of a three-dimensional gait signal-based geriatric cognitive impairment diagnosis device according to an embodiment of the present disclosure;

FIG. 3A and FIG. 3B illustrate a method of measuring a three-dimensional gait signal by using each measurement unit, according to an embodiment of the present disclosure;

FIG. 4A and FIG. 4B are graphs for comparing distribution results of probability values according to a geriatric cognitive impairment diagnosis device according to the present disclosure and a theoretical logistic function;

FIG. 5 and FIG. 6 are diagrams illustrating verification results of a geriatric cognitive impairment diagnosis device according to the present disclosure using actual geriatric cognitive impairment confirmation results; and

FIG. 7 is a flowchart illustrating a three-dimensional gait signal-based geriatric cognitive impairment diagnosis method according to another embodiment of the present disclosure.

### DETAILED DESCRIPTION OF THE EMBODIMENTS

Hereinafter, embodiments of the present disclosure will be described in detail with reference to the attached drawings such that those skilled in the art to which the present disclosure belongs may easily practice the present disclosure. However, the present disclosure may be implemented in various different forms and is not limited to the embodiments described herein. In addition, in order to clearly describe the present disclosure in the drawings, parts that are not related to the description are omitted, and similar components are given similar reference numerals throughout the specification.

In the entire specification of the present disclosure, when a component is described to be “connected” to another component, this includes not only a case where the component is “directly connected” to another component but also a case where the component is “electrically connected” to another component with another element therebetween.

Hereinafter, embodiments of the present disclosure will be described in detail with reference to the attached drawings.

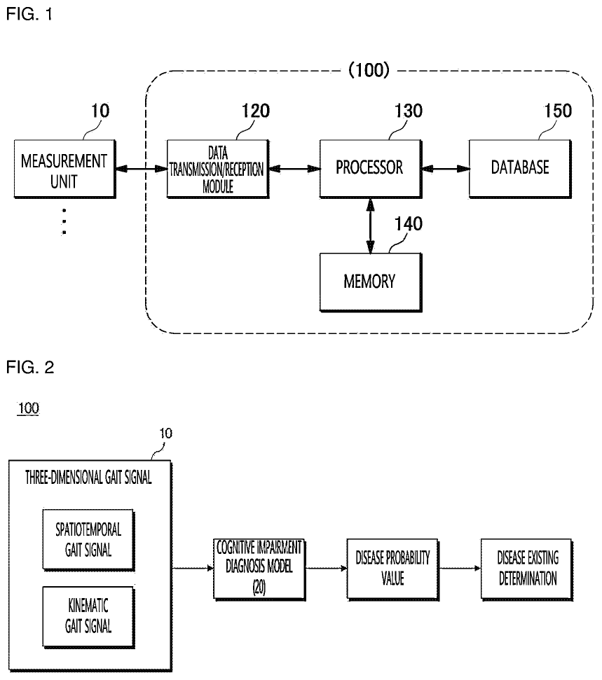

FIG. 1 is a configuration diagram of a three-dimensional (3D) gait signal-based geriatric cognitive impairment diagnosis device according to an embodiment of the present disclosure.

As illustrated in FIG. 1, a geriatric cognitive impairment diagnosis device 100 may include a data transmission/reception module 120, a processor 130, a memory 140, and a database 150.

The data transmission/reception module 120 may receive a three-dimensional gait signal including a spatiotemporal gait signal and a kinematic gait signal from each measurement unit 10 and transmit the three-dimensional gait signal to the processor 130.

The data transmission/reception module 120 may be a device including hardware and software required to transmit and receive signals, such as a control signal and a data signal through wired or wireless connections with other network devices.

Each three-dimensional gait signal transmitted to the data transmission/reception module 120 includes a spatiotemporal gait measurement value and a kinematic gait measurement value. For example, the spatiotemporal gait measurement value includes a stride length and a gait speed of an examinee during a gait cycle. In addition, the kinematic gait measurement value includes angles of the ankle, knee, and hip joints of an examinee, tilts of the front, back, left, and right of the pelvis, and accelerations applied to a lower body including the feet, thighs, and shins.

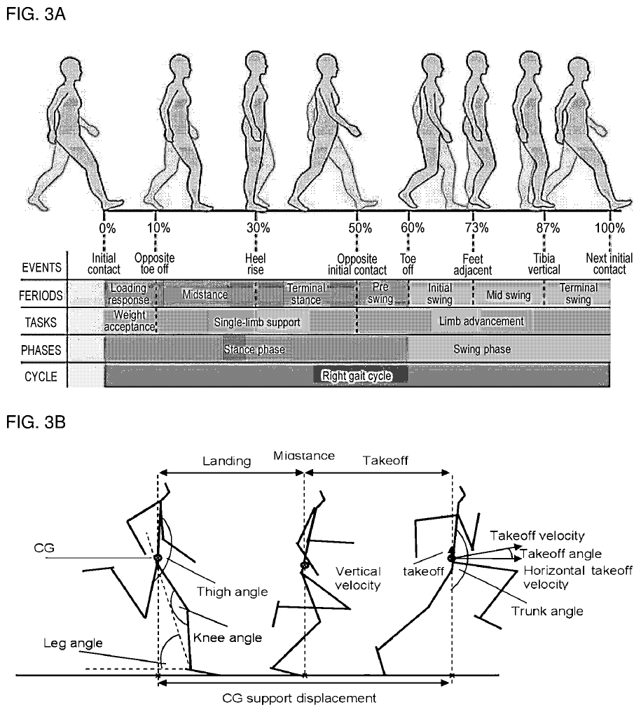

In this way, each measurement unit 10 may collect spatiotemporal gait measurement values and kinematic gait measurement values from an examinee, and transmit respective collected 3D gait signals to the data transmission/reception module 120. In this case, a method by which each measurement unit 10 measures a 3D gait signal of an examinee is described below with reference to FIG. 3A and FIG. 3B.

The processor 130 executes a cognitive impairment diagnosis program stored in the memory 140, and performs the following processing according to the execution of the cognitive impairment diagnosis program.

The program collects a three-dimensional gait signal including a spatiotemporal gait signal and a kinematic gait signal of an examinee, calculates a cognitive impairment disease probability value by using a cognitive impairment diagnosis model based on the three-dimensional gait signal, and determines whether there is a cognitive impairment disease based on the calculated probability value.

Therefore, the present disclosure has an effect of providing a diagnosis of geriatric cognitive impairment (for example, dementia and mild cognitive impairment) with high accuracy by using a kinematic gait signal in addition to the existing spatiotemporal gait signal.

The processor 130 may include all kinds of devices capable of processing data. For example, the processor 130 may be a data processing device that is built in hardware and has a physically structured circuit to perform a function represented by a code or command included in cognitive impairment diagnosis the program. For example, the data processing device built in the hardware may include a microprocessor, a central processing unit (CPU), a processor core, a multiprocessor, an application-specific integrated circuit (ASIC), a field programmable gate array (FPGA), or so on, but the scope of the present disclosure is not limited thereto.

The cognitive impairment diagnosis program is stored in the memory 140. The memory 140 stores an operating system for operating the geriatric cognitive impairment diagnosis device 100 or various types of data generated during execution of the cognitive impairment diagnosis program.

In this case, the memory 140 refers to a nonvolatile storage device that maintains the stored information even when power is not supplied and a volatile storage device that requires power to maintain the stored information.

In addition, the memory 140 may perform a function of temporarily or permanently storing the data processed by the processor 130. Here, the memory 140 may include magnetic storage media or flash storage media in addition to the volatile storage device that requires power to maintain the stored information, but the scope of the present disclosure is not limited thereto.

The database 150 stores or provides data required for the geriatric cognitive impairment diagnosis device 100 under the control of the processor 130. For example, the database 150 may store the probability of a cognitive impairment disease detected by using a cognitive impairment diagnosis model 20 based on the three-dimensional gait signal. The database 150 may be included as a separate component from the memory 140, or may be constructed in a part of the memory 140.

FIG. 2 is a structural diagram of a 3D gait signal-based geriatric cognitive impairment diagnosis device according to an embodiment of the present disclosure. FIG. 3A and FIG. 3B illustrate a method of measuring a 3D gait signal according by using each measurement unit to, according to an embodiment of the present disclosure.

Specifically, referring to FIG. 2, the geriatric cognitive impairment diagnosis device 100 collects a 3D gait signal including a spatiotemporal gait signal and a kinematic gait signal of an examinee, calculates a cognitive impairment disease probability value by using the cognitive impairment diagnosis model 20 based on the 3D gait signal, and executes a program that determines whether there is a cognitive impairment disease based on the calculated probability value.

For example, referring to FIG. 3A, the measurement unit 10 may measure a numerical value and graph obtained by three-dimensionally analyzing movements of the pelvis, hip joint, knee joint, and ankle joint of an examinee during a gait cycle in the front, side, and cross-sections as a spatiotemporal gait signal. Accordingly, a spatiotemporal gait measurement value may include a stride length and gait speed. In addition, values obtained by quantitatively measuring respective elements include quantitative proportional values including the stride length and gait speed. For example, the spatiotemporal gait measurement value may include the quantitative proportional values of the stride length and gait speed, such as stride time: 515.7, long stride time: 1012.9, steps per minute: 118.5, stride: 169.4, and gait speed: 6.1.

Referring to FIG. 3B, the measurement unit 10 may measure angles of the ankle, knee, and hip joints of an examinee, tilts of the front, back, left, and right of the pelvis, and accelerations applied to a lower body including the feet, thighs, and shins during a gait cycle as a kinematic gait signal. Accordingly, the kinematic gait measurement value may include an angle of a joint, an inclination of the pelvis, and an acceleration applied to various parts of the lower body. For example, the measurement unit 10 may include a sensor that is attached to joints of the lower body of an examinee in a natural and laboratory-based environment and measures anatomical joint angles, directions, and linear accelerations. For example, the sensor may be composed of an inertial measurement unit (IMU), and inertial measurement data and the movement of the examinee may be displayed in a graph in real time. For example, kinematic measurement values may include joint angles, directions, and linear accelerations, such as hip flexion (degree): 0.362, hip adduction (degree): −5.984, hip abduction (degree): −5.142, knee flexion (degree): 9.104, ankle dorsiflexion (degree): −5.877, ankle adduction (degree): −8.146, pelvic tilt (degree): 3.571, X-axis acceleration of foot (mG): −2235.398, Y-axis acceleration of foot (mG): −393.514, and Z-axis acceleration of foot (mG): 798.811.

The cognitive impairment diagnosis model 20 is a model constructed by applying a 3D gait signal of a patient with a geriatric cognitive impairment disease to a logistic function, and is obtained through a process of determining an estimated amount for constituting a logistic function based on actual data of a patient with a geriatric cognitive impairment disease. The cognitive impairment diagnosis model 20 includes a logistic function for calculating the probability of a geriatric cognitive impairment disease by using variables including spatiotemporal gait measurement values and kinematic gait measurement values.

In addition, the logistic function may further include demographic variables as the variables. For example, the demographic variables may include an age, gender, and education level. For example, the demographic variables may be defined as: late-elderly: 75 years or older (1), gender: female (0), and education: high school graduate or lower (0).

For example, the cognitive impairment diagnosis model 20 may calculate a statistic amount according to Equation 1, and estimate a relationship between the collected 3D gait signals (spatiotemporal gait measurement values and kinematic gait measurement values) and geriatric cognitive impairment according to a logistic function defined in Equation 2. That is, the estimated relationship is generated as an estimated value β for each variable. Next, an actual measured measurement value x and the estimated amount β for each variable are multiplied, and then all values are added.

$$Y = {\sum }_{i = 1}^{n} {β}_{i} {X}_{{GaitST}_{i} +} {\sum }_{j = 1}^{n} {β}_{j} {X}_{{GaitKN}_{i} +} {\sum }_{k = 1}^{n} {β}_{k} {X}_{{DEMO}_{k}} \qquad Equation 1$$

Here, GaitST is a spatiotemporal gait measurement value, GaitKN is a kinematic gait measurement value, DEMO is a demographic variable, and p is an estimated amount for each variable.

$$ln ( \frac{P}{1 - P} ) = {\sum }_{i = 1}^{n} {β}_{i} {X}_{{GaitST}_{i} +} {\sum }_{j = 1}^{n} {β}_{j} {X}_{{GaitKN}_{i} +} {\sum }_{k = 1}^{n} {β}_{k} {X}_{{DEMO}_{k}} \qquad Equation 2$$

Here, P is a disease probability value that an examinee (patient) has senile cognitive impairment (disease), and has a value between 0 and 1.

Next, the cognitive impairment diagnosis model 20 may calculate the disease probability value P according to Equation 3 and Equation 4. First, a summed value according to Equation 2 is exponentially transformed according to Equation 3. Thereafter, the summed value is further transformed according to Equation 4 to calculate the disease probability value P.

$$( \frac{P}{1 - P} ) = {e}^{{\sum }_{i = 1}^{n} {β}_{i} {X}_{{GaitST}_{i} +} {\sum }_{j = 1}^{n} {β}_{j} {X}_{{GaitKN}_{i} +} {\sum }_{k = 1}^{n} {β}_{k} {X}_{{DEMO}_{k}}} \qquad Equation 3$$

$$P = \frac{{e}^{{\sum }_{i = 1}^{n} {β}_{i} {X}_{{GaitST}_{i} +} {\sum }_{j = 1}^{n} {β}_{j} {X}_{{GaitKN}_{i} +} {\sum }_{k = 1}^{n} {β}_{k} {X}_{{DEMO}_{k}}}}{1 - {e}^{{\sum }_{i = 1}^{n} {β}_{i} {X}_{{GaitST}_{i} +} {\sum }_{j = 1}^{n} {β}_{j} {X}_{{GaitKN}_{i} +} {\sum }_{k = 1}^{n} {β}_{k} {X}_{{DEMO}_{k}}}} \qquad Equation 4$$

That is, by Equation 3 and Equation 4, a transformed result value is determined as a final disease probability value P.

Thereafter, the program determines whether there is a cognitive disorder disease based on the disease probability value P calculated by the cognitive disorder diagnosis model 20. For example, as defined in Equation 5, when the result value is less than 1 and greater than 0.5, the examinee may be diagnosed to have a cognitive disorder disease, and when the result value is greater than or equal to 0 and less than or equal to 0.5, the examinee may be diagnosed to be normal.

$$decision ( x ) = \{ \begin{matrix}1 ifP ( y = 1 \vert x ) > 0.5 \\ 0 otherwise\end{matrix} \qquad Equation 5$$

In other words, as shown in FIG. 2, the geriatric cognitive impairment diagnosis device 100 inputs the collected 3D gait signal of the examinee to the cognitive impairment diagnosis model 20, and the cognitive impairment diagnosis model 20 calculates the disease probability value P based on measurement values and estimated amounts for each independent variable (spatiotemporal gait measurement values, kinematic gait measurement values, and demographic variables) by using Equation 1 to Equation 4 described above. In addition, it is possible to determine whether the examinee has geriatric cognitive impairment disease based on the disease probability value P by using Equation 5.

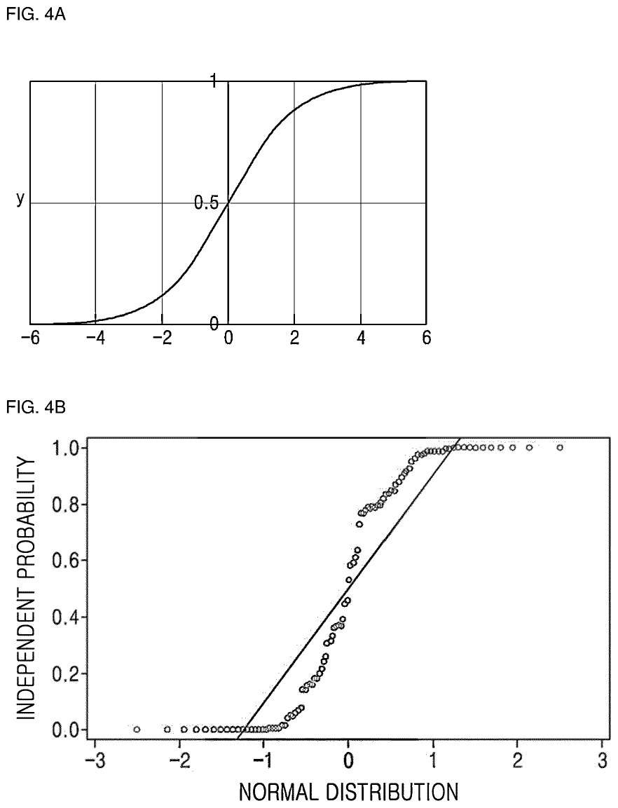

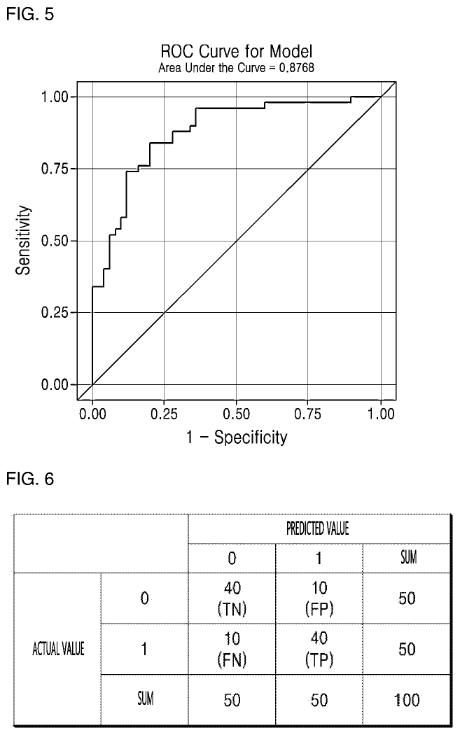

FIGS. 4A and 4B are graphs for comparing distribution results of probability values according to a geriatric cognitive impairment diagnosis device according to the present disclosure and a theoretical logistic function, and FIGS. 5 and 6 are diagrams illustrating verification results of the geriatric cognitive impairment diagnosis device according to the present disclosure using actual geriatric cognitive impairment confirmation results.

FIG. 4A is a graph illustrating the distribution of probability values of a theoretical logistic function, and FIG. 4B illustrates a distribution result of probability values of geriatric cognitive impairment obtained from 100 elderly people aged 65 or older by using the geriatric cognitive impairment diagnosis device 100 of the present disclosure.

As illustrated in FIG. 4A and FIG. 4B, distribution results of probability values of the geriatric cognitive impairment diagnosis device 100 based on measured values of 100 examinees and estimated values show a very similar pattern to a theoretical distribution of a logistic function.

FIG. 5 illustrates a result of applying the geriatric cognitive impairment diagnosis device 100 to 100 elderly people aged 65 or older, and shows a receiver-operating characteristic (ROC) curve obtained from geriatric cognitive impairment diagnosis. In this case, an area under the ROC curve is 0.8768 and shows excellent prediction performance.

FIG. 6 is a table showing accuracy of the geriatric cognitive impairment diagnosis device 100 of the present disclosure by crossing a geriatric cognitive impairment diagnosis value (actual value) diagnosed by clinical means using a confusion matrix and a geriatric cognitive impairment diagnosis value (predicted value) according to the present disclosure.

In this case, the accuracy is calculated as Accuracy=(TP+TN)/(TP+FP+TN+FN)=(40+40)/(40+10+40+10)=0.80, and the calculated accuracy of the disease diagnosis is represented to 80%.

As such, it can be seen that a disease diagnosis result of the geriatric cognitive impairment diagnosis device 100, to which a logistic function of the present disclosure is applied, is highly reliable.

Hereinafter, descriptions of the same configuration as the configurations illustrated in FIGS. 1 to 6 described above are omitted.

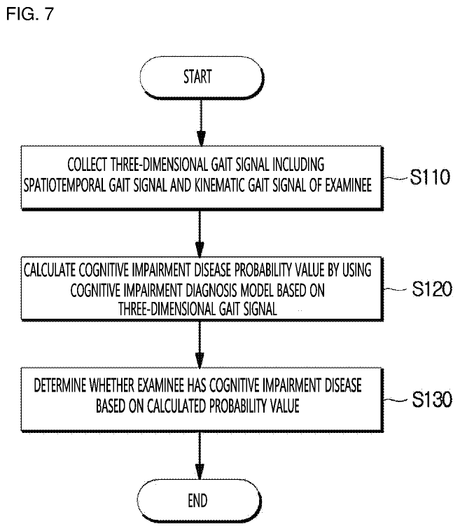

FIG. 7 is a flowchart illustrating a 3D gait signal-based geriatric cognitive impairment diagnosis method according to another embodiment of the present disclosure.

Referring to FIG. 7, a 3D gait signal-based geriatric cognitive impairment diagnosis method according to another embodiment of the present disclosure, which uses the 3D gait signal-based senile cognitive impairment diagnosis device 100, includes step S110 of collecting a 3D gait signal including a spatiotemporal gait signal and a kinematic gait signal of an examinee, step S120 of calculating a cognitive impairment disease probability value by using a cognitive impairment diagnosis model based on the 3D gait signal, and step S130 of determining whether there is a cognitive impairment disease based on the calculated probability value. In this case, the cognitive impairment diagnosis model is a model constructed by applying a 3D gait signal of a patient with geriatric cognitive impairment disease to a logistic function.

In step S110, a spatiotemporal gait measurement value including a stride length and a gait speed measured during a gait cycle of an examinee may be calculated by the measurement unit 10, and a kinematic gait measurement value including angles of the ankle, knee, and hip joints of the examinee, tilts of the front, back, left, and right of the pelvis, and accelerations applied to a lower body including the feet, thighs, and shins may be calculated.

The cognitive impairment diagnosis model 20 includes a logistic function for calculating a probability value of a geriatric cognitive impairment disease by using variables including the spatiotemporal gait measurement value and the kinematic gait measurement value.

In step S120, the cognitive impairment diagnosis model 20 may estimate a relationship between a 3D gait signal measured by the measurement unit 10 and a geriatric cognitive impairment according to a logistic function previously defined as in Equation 1 to Equation 4 described above, and may generate the estimated value p to be assigned to each collected measurement value. In addition, an actual measured value x may multiplied by the estimated value p for each variable (the spatiotemporal gait measurement value and the kinematic gait measurement value) and then all values may be summed. Subsequently, the summed value may be subjected to exponential transformation and additional transformation to calculate the disease probability value P.

Next, in step S130, whether the examinee has a cognitive impairment disease may be determined based on the calculated disease probability value P, and when the disease probability value P calculated by the predefined Equation 5 is less than 1 and greater than 0.5, it can be diagnosed that the examinee has a cognitive impairment disease, and when the disease probability value P calculated by the predefined Equation 5 is greater than 0 and less than or equal to 0.5, it can be diagnosed that the examinee is normal.

Embodiments of the present disclosure may be performed in the form of a recording medium including instructions executable by a computer, such as a program module executed by a computer. A computer readable medium may be any available medium that may be accessed by a computer and includes both volatile and nonvolatile media, removable and non-removable media. Also, the computer readable medium may include a computer storage medium. A computer storage medium includes both volatile and nonvolatile media and removable and non-removable media implemented by any method or technology for storing information, such as computer readable instructions, data structures, program modules or other data.

In addition, although a method and device of the present disclosure are described with respect to specific embodiments, some or all of components or operations thereof may be implemented by using a computer system having a general-purpose hardware architecture.

The above description of the present disclosure is for illustrative purposes only, and those skilled in the art to which the present disclosure belongs will understand that the present disclosure may be easily modified into another specific form based on the descriptions given above without changing the technical idea or essential features of the present disclosure. Therefore, the embodiments described above should be understood as illustrative in all respects and not limiting. For example, each component described as single may be implemented in a distributed manner, and similarly, components described as distributed may also be implemented in a combined form.

The scope of the present disclosure is indicated by the claims described below rather than the detailed description above, and all changes or modified forms derived from the meaning and scope of the claims and their equivalent concepts should be construed as being included in the scope of the present disclosure.

What is claimed is:

1. A three-dimensional gait signal-based geriatric cognitive impairment diagnosis device comprising:
a data transmission/reception module;
a memory storing a geriatric cognitive impairment diagnosis program; and
a processor configured to execute the geriatric cognitive impairment diagnosis program stored in the memory,
wherein the geriatric cognitive impairment diagnosis program collects a three-dimensional gait signal including a spatiotemporal gait signal and a kinematic gait signal of an examinee, calculates a cognitive disorder disease probability value by using a cognitive disorder diagnosis model based on the three-dimensional gait signal, and determines whether the examinee has a cognitive disorder disease based on the calculated cognitive disorder disease probability value, and
the cognitive disorder diagnosis model is constructed by applying the three-dimensional gait signal of a patient with a geriatric cognitive disorder disease to a logistic function.

2. The three-dimensional gait signal-based geriatric cognitive impairment diagnosis device of claim 1, wherein
the geriatric cognitive impairment diagnosis program calculates a spatiotemporal gait measurement value including a stride length and a gait speed measured during a gait cycle of the examinee by using a measurement unit, and calculates a kinematic gait measurement value including angles of an ankle, a knee, and a hip joint of the examinee, tilts of a front, a back, a left side, and a right side of a pelvis, and an acceleration applied to a lower body including a foot, a thigh, and a shin.

3. The three-dimensional gait signal-based geriatric cognitive impairment diagnosis device of claim 2, further comprising:
a logistic function that calculates a geriatric cognitive impairment disease probability value by using variables including the spatiotemporal gait measurement value and the kinematic gait measurement value.

4. A geriatric cognitive impairment diagnosis method which is performed by a three-dimensional gait signal-based geriatric cognitive impairment diagnosis device, the geriatric cognitive impairment diagnosis method comprising:
collecting a three-dimensional gait signal including a spatiotemporal gait signal and a kinematic gait signal of an examinee;
calculating a cognitive impairment disease probability value by using a cognitive impairment diagnosis model based on the three-dimensional gait signal; and
determining whether the examinee has a cognitive impairment disease based on the calculated cognitive impairment disease probability value,
wherein the cognitive impairment diagnosis model is constructed by applying a three-dimensional gait signal of a patient with a geriatric cognitive impairment disease to a logistic function.

5. The geriatric cognitive impairment diagnosis method of claim 4, wherein, in the collecting of the three-dimensional gait signal,
a spatiotemporal gait measurement value including a stride length and a gait speed measured during a gait cycle of the examinee is calculated by a measurement unit, and
a kinematic gait measurement value is calculated which includes angles of an ankle, a knee, and a hip joint of the examinee, tilts of a front, a back, a left side, and a right side of a pelvis, and an acceleration applied to a lower body including a foot, a thigh, and a shin.

6. The geriatric cognitive impairment diagnosis method of claim 5, wherein the cognitive impairment diagnosis model includes a logistic function that calculates a geriatric cognitive impairment disease probability value by using variables including the spatiotemporal gait measurement value and the kinematic gait measurement value.

7. A non-transitory computer-readable recording medium on which a computer program for performing a three-dimensional gait signal-based geriatric cognitive impairment diagnosis method according to claim 4 is recorded.
